(19) United States
(12) Patent Application Publication
(10) Pub. No.: US 2017/0020776 A1
(43) Pub. Date: Jan. 26, 2017

(54) RESPIRATORY THERAPY SYSTEMS, SENSORS ARRANGEMENTS AND METHODS

(71) Applicant: SMITHS MEDICAL INTERNATIONAL LIMITED, Ashford, Kent (GB)

(72) Inventors: Mohammad Qassim Mohammad Khasawneh, Luton (GB); Mark Charles Oliver, Hertfordshire (GB); Mark Sinclair Varney, Bedfordshire (GB)

(73) Assignee: SMITHS MEDICAL INTERNATIONAL LIMITED, Ashford, Kent (GB)

(21) Appl. No.: 15/113,557

(22) PCT Filed: Dec. 19, 2014
(86) PCT No.: PCT/GB2014/000521

§ 371 Date: Jul. 22, 2016

(30) Foreign Application Priority Data
Jan. 30, 2014 (GB) 1401566.3

(51) Int. Cl. A61H 23/02; A61B 5/08; A61B 5/085; A61B 5/00

(52) U.S. Cl. CPC A61H 23/02; A61B 5/6823; A61B 5/0803; A61B 5/085; A61B 5/486; A61B 2562/046; A61H 2205/084

(57) ABSTRACT

A respiratory therapy system includes a vibratory expiratory therapy device through which the patient exhales to set up vibrations within the chest. An array of piezoelectric vibration sensors is mounted on the patient's chest and supplies vibration outputs to a processor, which in turn provides an output to a display indicative of impedance of different regions of the chest.

This invention relates to sensor arrangements of the kind for providing information about the condition of a patient's lungs.

The invention is more particularly, but not exclusively, concerned with respiratory therapy systems including a device that provides an alternating, vibratory resistance to breathing through the apparatus and sensing means responsive to vibration produced in the chest.

Patients with respiratory system diseases, such as asthma, COPD, cystic fibrosis and the like, have a prominent pathophysiological feature in the form of hyper secretion of mucus, often accompanied by impaired mucus transport. This imbalance between mucus transport and secretion results in mucus being retained in the respiratory system. Positive expiratory pressure (PEP) apparatus, that is, apparatus that presents a resistance to expiration through the device, are now widely used to help treat patients suffering from a range of respiratory impairments. More recently, such apparatus that apply chest physiotherapy by providing an alternating resistance to flow have been found to be particularly effective. One example of such apparatus is sold under the trade mark Acapella (a registered trade mark of Smiths Medical) by Smiths Medical and is described in U.S. Pat. No. 6,581,598, U.S. Pat. No. 6,776,159, U.S. Pat. No. 7,059,324 and U.S. Pat. No. 7,699,054. Other vibratory respiratory therapy (V-PEP) apparatus is available, such as “Quake” manufactured by Thayer, “AeroPEP” manufactured by Monaghan, “TheraPEP” manufactured by Smiths Medical and “TV Percussionator” manufactured by Percussionaire Corp. The generated vibratory positive pressures mechanically reduce the viscoelasticity of sputum by breaking down the bonds of mucus macromolecules which enhances mucociliary clearance. Alternative apparatus such as “CoughAssist” manufactured by Philips are also available. Respiratory therapy apparatus can instead provide an alternating resistance to flow during inhalation.

Although these devices can be very effective, users often neglect to use them correctly or do not use them regularly at the prescribed frequency. It is very difficult to maintain a record of use of the device, especially when the patient is using it at home. The clinician often does not know whether deterioration in a patient's condition is because he has failed to use the device as prescribed or whether other factors are the cause. The effectiveness of treatment by such V-PEP devices is also critically dependent on the frequency and amplitudes on the generated vibration. Although there have been proposals to monitor operation of such devices these proposals have not addressed how to measure actual vibration within the lung. Information about the condition of a patient's lungs is difficult to obtain especially as regards the impedance to transmission of pressure waves in different regions.

It is an object of the present invention to provide alternative sensor arrangements, respiratory therapy systems and methods of deriving information about the condition of a patient's lungs.

According to one aspect of the present invention there is provided a sensor arrangement of the above-specified kind, characterised in that the arrangement includes a plurality of vibration sensors arranged for mounting at spaced locations on the patient's chest to provide an output indicative of vibration at different locations within the chest and a processor connected to receive the outputs from the sensors, and that the processor is arranged to provide an output indicative of the condition of the patient's lungs.

The sensors are preferably arranged in a grid of rows for mounting laterally across the chest and columns for mounting longitudinally along the chest. The sensors are preferably arranged to have maximum sensitivity to vibration in a direction orthogonally to the plane of the wall of the chest. The vibration sensors may be piezoelectric film sensors. The output indicative of the condition of the patient's lungs preferably includes information about the impedance of the chest in different regions.

According to another aspect of the present invention there is provided a respiratory therapy system including a vibratory respiratory therapy device of the kind through which a patient breathes in order to set up vibrations in his chest, and a sensor arrangement according to the above one aspect of the present invention, characterised in that the sensor arrangement is responsive to vibration in the chest caused by use of the respiratory therapy device.

According to a further aspect of the present invention there is provided a respiratory therapy system including a vibratory respiratory therapy device of the kind through which a patient breathes in order to set up vibrations in his chest, characterised in that the system includes a plurality of vibration sensors arranged for mounting on the patient's torso at spaced locations to be responsive to vibration in the chest caused by use of the therapy device and a processor connected to receive outputs from the sensors and arranged to provide an output indicative of the condition of the patient's lungs.

The respiratory therapy device is preferably an expiratory therapy device. The therapy device may include a rocker arm arranged to open and close an outlet during exhalation.

According to a fourth aspect of the present invention there is provided a method of deriving information about the condition of a patient's lungs including the steps of mounting a plurality of vibration sensors at spaced locations on the torso of the patient, the vibration sensors being connected with a processor, having the patient breathe through a vibratory respiratory therapy device so that vibrations are set up in the patient's chest, and deriving an indication of the condition of the patient's chest from the processor in accordance with the outputs from the sensors.

A system including a vibratory respiratory therapy device will now be described, by way of example, with reference to the accompanying drawings, in which:

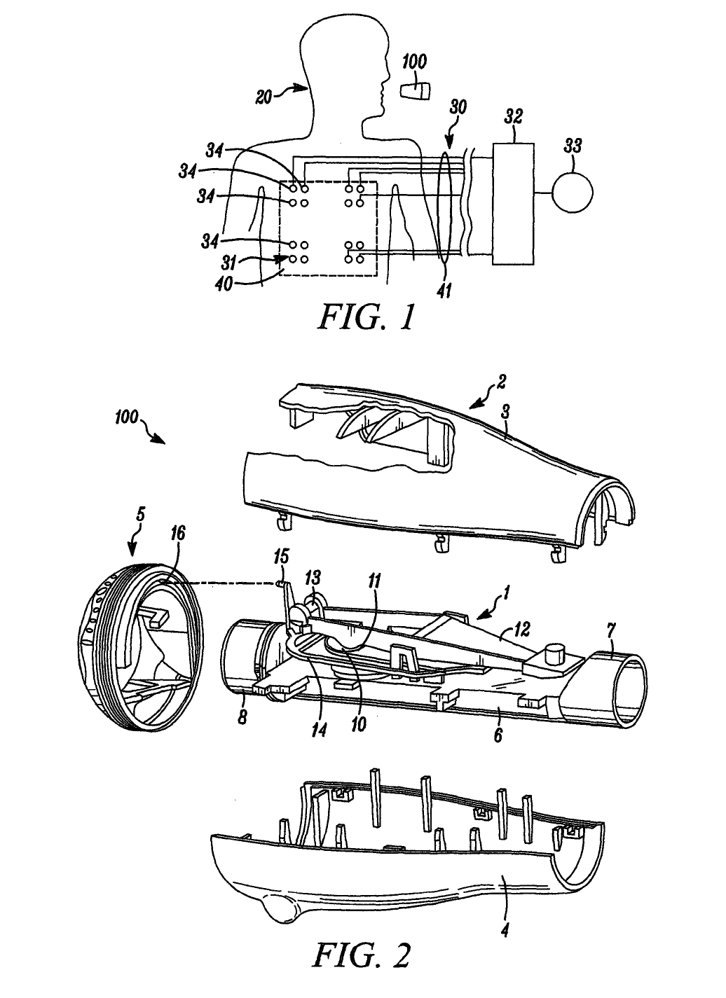

FIG. 1 illustrates the system in use; and

FIG. 2 is an exploded view of the respiratory therapy device;

With reference first to FIG. 1 there is shown a patient 20, a respiratory therapy device 100 and sensor apparatus 30 responsive to vibration in the lungs of the patient caused by use of the device. The therapy device 100 and sensor apparatus 30 together provide a respiratory therapy system.

The respiratory therapy device may be of any conventional kind that produces vibration within the user's lungs. The device 100 shown in FIG. 2 is an Acapella respiratory therapy device as sold by Smiths Medical. The device 100 comprises a rocker assembly 1 contained within an outer housing 2 provided by an upper part 3 and a lower part 4 of substantially semi-cylindrical shape. The device is completed by an adjustable dial 5 of circular section. The rocker assembly 1 includes an air flow tube 6 with a breathing inlet 7 at one end and an inspiratory inlet 8 at the opposite end including a one-way valve (not shown) that allows air to flow into the air flow tube 6 but prevents air flowing out through the inspiratory inlet. The air flow tube 6 has an outlet opening 10 with a non-linear profile that is opened and closed by a conical valve element 11 mounted on a rocker arm 12 pivoted midway along its length about a transverse axis. The air flow tube 6 and housing 2 provide a structure with which the rocker arm 12 is mounted. At its far end, remote from the breathing inlet 7, the rocker arm 12 carries an iron pin 13 that interacts with the magnetic field produced by a permanent magnet (not visible) mounted on an adjustable support frame 14. The magnet arrangement is such that, when the patient is not breathing through the device, the far end of the rocker arm 12 is held down such that its valve element 11 is also held down in sealing engagement with the outlet opening 10. A cam follower projection 15 at one end of the support frame 14 locates in a cam slot 16 in the dial 5 such that, by rotating the dial, the support frame 14, with its magnet, can be moved up or down to alter the strength of the magnetic field interacting with the iron pin 13. The dial 5 enables the frequency of operation and the resistance to flow of air through the device to be adjusted for maximum therapeutic benefit to the user.

When the patient inhales through the breathing inlet 7 air is drawn through the inspiratory inlet 8 and along the air flow tube 6 to the breathing inlet. When the patient exhales, the one-way valve in the inspiratory inlet 8 closes, preventing any air flowing out along this path. Instead, the expiratory pressure is applied to the underside of the valve element 11 on the rocker arm 12 causing it to be lifted up out of the opening 10 against the magnetic attraction, thereby allowing air to flow out to atmosphere. The opening 10 has a non-linear profile, which causes the effective discharge area to increase as the far end of the rocker arm 12 lifts, thereby allowing the arm to fall back down and close the opening. As long as the user keeps applying sufficient expiratory pressure, the rocker arm 12 will rise and fall repeatedly as the opening 10 is opened and closed, causing a vibratory, alternating or oscillating resistance to expiratory breath flow through the device. Further information about the construction and operation of the device can be found in U.S. Pat. No. 6,581,598, the contents of which are hereby incorporated into the present application.

With reference now to FIG. 1, the sensor apparatus 30 includes a sensor arrangement 31, a processor 32 and display or utilisation means 33. The sensor arrangement 31 includes at least two vibration sensors 34, such as piezoelectric film sensors, that are responsive primarily to vibration in a direction orthogonal to the surface of the chest wall caused by pressure waves in the chest during use of the therapy device 100. The vibration sensors 34 are spaced from one another at different locations on the patient's torso and, more particularly, on his chest wall. Preferably the sensor arrangement 31 includes a two dimensional array or grid of sensors (as shown) arranged in rows (such as of four sensors) laterally across the chest wall and in columns (such as of ten sensors) extending longitudinally of the chest. The sensors 34 are preferably positioned over the lungs, such as shown in FIG. 2 where there are four columns of sensors arranged with two columns on each lateral side of the chest. The sensors 34 may be individually mounted on the chest wall or they could be attached to a common flexible, adhesive mat 40 so that all the sensors can be attached to the patient at the same time. Wires 41 extend from the sensors 34 to the processor 32, which may be supported on the patient, such as on a belt, or may be mounted nearby on a pole or table. Alternatively, the sensors 34 could connect with a wireless transmitter (not shown) such as an infra-red or radio frequency transmitter carried by the patient so that data from the sensors can be transmitted to the processor wirelessly. FIG. 1 shows the sensor arrangement 31 attached to the chest wall on the anterior surface of the patient's torso but it would be possible to attach a sensor arrangement to other parts of the torso, such as on the patient's back either instead of or in addition to the sensor arrangement on the chest wall.

The vibration sensors 34 provide electrical outputs in accordance with the sensed vibration caused by pressure waves within the chest. By arranging the sensors 34 in a two dimensional array it is possible to obtain a two dimensional image of the vibration pattern within the chest whereas the timing or phase information of the vibration received by each sensor gives depth information. This information combined, therefore, enables a three dimension image to be obtained of vibration within the chest showing how the impedance to the transmission of pressure waves varies through the chest.

The processor 32 carries out appropriate filtering and analysis of the output of the sensors 34 and provides information to the utilisation means 33, which may be a display or data store, indicative of the condition of the patient's lungs in different regions. In particular the information may include information about the impedance of different regions, information about obstruction or restriction to airflow and the like.

The processor 32 could be arranged to prompt the patient 20 to vary his use of the therapy device 100, such as by varying the frequency or intensity of the vibrations produced. By monitoring the vibration at these different frequencies or intensities the system would be able to obtain additional data about the efficiency of the transmission of pressure waves through the lungs under different conditions for use in deriving enhanced diagnostic information.

Where a respiratory therapy device 100 is used this could be arranged to provide an output indicative of a function of the device, such as positive expired pressure, which is supplied to the processor 32 to provide additional diagnostic information.

It is not essential that the system include a vibratory therapy device since vibration or pressure waves in the chest could be caused by some other pressure wave generator. The pressure wave generator could be provided by an acoustic transmitter positioned in an acoustic path to the airway, such as in a short tube extending from the patient's mouth.

The system could alternatively be used without any pressure wave generator simply by monitoring lung sounds produced during normal breathing by the patient.

The system could be arranged to derive an output representative of heart sounds, to replace the need for a separate heart rate monitor. The vibration produced by the heart could be used as a source of pressure waves to provide additional or alternative information about lung impedance.

The invention is not confined to use with expiratory therapy devices but could be useful also in inspiratory vibratory therapy.

1-10. (canceled)

11. A sensor arrangement for providing information about the condition of a patient's lungs, characterized in that the arrangement includes a plurality of vibration sensors arranged for mounting at spaced locations on the patient's chest to provide an output indicative of vibration at different locations within the chest and a processor connected to receive the outputs from the sensors, and that the processor is arranged to provide an output indicative of the condition of the patient's lungs.

12. A sensor arrangement according to claim 11, characterized in that the sensors are arranged in a grid of rows for mounting laterally across the chest and columns for mounting longitudinally along the chest.

13. A sensor arrangement according to claim 11, characterized in that the sensors are arranged to have maximum sensitivity to vibration in a direction orthogonally to the plane of the wall of the chest.

14. A sensor arrangement according to claim 11, characterized in that the vibration sensors are piezoelectric film sensors.

15. A sensor arrangement according to claim 11, characterized in that the output indicative of the condition of the patient's lungs includes information about the impedance of the chest in different regions.

16. A respiratory therapy system including a vibratory respiratory therapy device of the kind through which a patient breathes in order to set up vibrations in his chest, and a sensor arrangement including a plurality of vibration sensors arranged for mounting at spaced locations on the patient's chest to provide an output indicative of vibration at different locations within the chest and a processor connected to receive the outputs from the sensors, wherein the sensor arrangement is responsive to vibration in the chest caused by use of the respiratory therapy device.

17. A respiratory therapy system according to claim 16, characterized in that the respiratory therapy device is an expiratory therapy device.

18. A respiratory therapy system according to claim 17, characterized in the therapy device includes a rocker arm arranged to open and close an outlet during exhalation.

19. A respiratory therapy system including a vibratory respiratory therapy device of the kind through which a patient breathes in order to set up vibrations in his chest, characterized in that the system includes a plurality of vibration sensors arranged for mounting on the patient's torso at spaced locations to be responsive to vibration in the chest caused by use of the therapy device and a processor connected to receive outputs from the sensors and arranged to provide an output indicative of the condition of the patient's lungs.

20. A respiratory therapy system according to claim 19, characterized in that the respiratory therapy device is an expiratory therapy device.

21. A respiratory therapy system according to claim 20, characterized in the therapy device includes a rocker arm arranged to open and close an outlet during exhalation.

22. A method of deriving information about the condition of a patient's lungs including mounting a plurality of vibration sensors at spaced locations on the torso of the patient, the vibration sensors being connected with a processor, having the patient breathe through a vibratory respiratory therapy device so that vibrations are set up in the patient's chest, and deriving an indication of the condition of the patient's chest from the processor in accordance with the outputs from the sensors.
